(19) United States
(12) Patent Application Publication
(10) Pub. No.: US 2006/0200103 A1
(43) Pub. Date: Sep. 7, 2006

(54) Solid regenerated standard viscose fibres

(76) Inventors: Josef Schmidtbauer, Vocklabruck (AT); Heinrich Schmidt, Vocklabruck (AT); Christoph Boxan, Lenzing (AT); Daniel Thomas Blair, Morristown, TN (US)

Correspondence Address: BAKER & BOTTS, 30 ROCKEFELLER PLAZA, 44TH FLOOR, NEW YORK, NY, 10112, US

(21) Appl. No.: 11/236,128

(22) Filed: Sep. 27, 2005

Related U.S. Application Data
(63) Continuation of application No. PCT/AT04/00074, filed on Mar. 8, 2004.

(30) Foreign Application Priority Data
Mar. 27, 2003 (AT) A 488/2003

(51) Int. Cl. A61F 13/15

(52) U.S. Cl. 604/358

(57) ABSTRACT

The present invention relates to a solid regenerated standard viscose fibre. The fibre according to the invention has a cross section the area of which is larger than the area of the largest equilateral triangle inscribed into said cross section by a factor of less than 2.50 times, preferably less than 2.40 times, especially preferred less than 2.25 times. Furthermore, the fibre according to the invention has a Syngina absorbency as defined hereinbefore of more than 6.0 g/g fibre.

### CROSS REFERENCE TO RELATED APPLICATIONS

This application is a continuation of International Patent Application No. PCT/AT2004/000074 filed Mar. 8, 2004, designating the United States and published in the German language on Oct. 7, 2004 under International Patent Publication No. WO 2004/085720, which is based on Austrian Patent Application No. A 488/2003 filed Mar. 27, 2003, to each of which priority is claimed, and each of which is incorporated by reference in its entirety herein.

### FEDERALLY FUNDED GRANT SUPPORT

Not applicable.

### 1. INTRODUCTION

The present invention relates to a solid regenerated standard viscose fibre and a process for the manufacture of said fibre.

### 2. BACKGROUND OF THE INVENTION

State-of-the-art fibre materials commonly used for the manufacturing of tampons are regular viscose fibres, so-called trilobal viscose fibres and cotton. The specific absorbency of these fibres according to the Syngina Test as described below is approximately 4.5 g/g for cotton, 5.5 g/g for regular viscose and 6.5 g/g for trilobal viscose fibres.

It is the aim of tampon manufacturers to obtain a certain level of absorbency with a minimum amount of fibre material and costs.

Whereas cotton is going to be phased out as a fibre material for tampons due to its insufficient absorbency, trilobal fibres are much more expensive to produce and much more difficult to process into tampons compared to regular viscose.

In order to enhance the absorbency of cellulosic fibres, many different approaches have been reported:
1. a chemical modification by grafting of monomers onto the cellulosic fibre,
2. a chemical modification by incorporation of absorbent polymers like carboxymethyl-cellulose, chitosan, cellulose carbamate, alginate or guaran into the cellulosic fibre matrix
3. a physical modification of the fibres such as hollow fibres or collapsed hollow fibres as known for example from U.S. Pat. No. 4,129,679 or
4. multilimbed fibres (so-called “trilobal” fibres) obtained by using spinnerets with multilimbed extrusion holes having at least 3 limbs with an aspect ratio of 2:1 to 10:1 as known for example from EP-A1 0 301 874.

The disadvantage of a chemical modification of the cellulosic fibre is, that a costly and time-consuming toxicological and physiological testing procedure is needed for very sensitive medical applications like tampons and that the occurrence of the Toxic Shock Syndrom (TSS) prevents most tampon manufacturers from using chemically modified fibre materials, although the chemicals may regarded as safe.

The disadvantage of hollow and collapsed hollow fibres is, that they are difficult to produce because of their high water retention value, which makes the fibres swell strongly during washing and stick together by formation of hydrogen bonds during drying, making them brittle in the dry state, soapy in the wet state and difficult to open and process into a carded web.

The use of trilobal fibres has continuously increased during the past couple of years, although trilobal fibres are much more difficult to process into a tampon. The small limbs of the fibres are very fragile and may easily be damaged by mechanical forces which are applied during processing of the fibres, especially during carding under formation of fibre dust.

The manufacture of multi-limbed viscose fibres has been described e.g. in U.S. Pat. Nos. 5,634,914 and 5,458,835 and EP-A1 0 301 874. The process disclosed therein describes the spinning of a commonly used viscose, which may contain a certain amount of a modifier known in the art through extrusion holes having a multi-limbed shape, especially a trilobal shape into a conventional spinbath. The essential feature of this process is that the shape of the multi-limbed extrusion holes in the spinneret is similar to the desired shape of the cross section of the filaments. According to the teaching of these documents, the geometry of the spinneret hole determines the shape of the fibre cross section and a certain aspect ratio of the fibre cross section can be obtained by a corresponding design of the extrusion holes.

The state of the art in regard to multi-limbed fibres, furthermore, teaches that such multi-limbed fibres have an increased absorbency compared to state-of-the-art viscose fibres, especially in tampons and that such fibres need to have at least 3 limbs and that each limb of such fibres needs to have an aspect ratio of at least 2:1, most preferably of 3:1 to 5:1. The higher the aspect ratio, the higher would be the degree of free volume and the absorbency of the fibres, provided that the limbs are not so long and thin that they bend back upon themselves.

As described in e.g., U.S. Pat. Nos. 5,634,914 and 5,458,835, the aspect ratio is the ratio of the length to the average width of the fibre limbs. This ratio may be calculated for each limb by taking one length measurement and three width measurements. The widths are measured at about the midpoint and at either end of the limb and then the average width of the limb is calculated from the three measurements.

It is also mentioned in these documents without further evidence, that even higher absorbencies of multilimbed fibres can be achieved under slow regeneration spinning conditions, e.g. by decreasing the acid level and/or increasing the sulphate level and/or addition of a viscose modifier.

The fact that concavities in the cross section of viscose fibres enhances the absorbency of these fibres and the products made therefrom is, furthermore, known from U.S. Pat. No. 4,362,159. In addition, U.S. Pat. No. 4,129,679 discloses that a multi-limbed cross section of the viscose fibres confers greater absorbency from the products made from the filaments by virtue of the capacity of the filament bundle to hold large quantities of interstitial water between adjacent limbs of filaments.

### 3. SUMMARY OF THE INVENTION

It has now surprisingly been found that it is possible to produce a viscose fibre from spinnerets with multi-limbed orifices, whereby the cross section of the fibre is of a substantially triangular shape not having limbs with an aspect ratio of less than 2:1. This fibre avoids the drawbacks of multi-limbed fibres, but surprisingly although not having a multi-limbed cross section, the fibre is highly absorbent.

Accordingly, the present invention relates to a solid regenerated standard viscose fibre having a cross section the area of which is larger than the area of the largest equilateral triangle inscribed into said cross section by a factor of less than 2.50 times, preferably less than 2.40 times, especially preferred less than 2.25 times, and having a Syngina absorbency as defined hereinafter of more than 6.0 g/g fibre.

### 4. BRIEF DESCRIPTION OF THE FIGURES

FIG. 1 shows a triangle inscribed into the cross section of a triangular fibre according to the present invention.

FIG. 2 shows a triangle inscribed into the cross section of a trilobal fibre according to the state of the art.

FIG. 3 depicts the apparatus used to perform the Syngina test method.

FIG. 4 depicts the mechanical press used to prepare the test specimen for the Syngina test method.

FIG. 5 is a sectional view of a component of the mechanical press of FIG. 4, according to lines A-A.

FIG. 6 is a sectional view of another component of the mechanical press of FIG. 4, according to lines B-B.

FIG. 7 is an enlarged sectional view of region Y in FIG. 5.

FIG. 8 is an enlarged sectional view of region Z in FIG. 6.

FIG. 9 to 15 show the shapes of the fibres produced according to Examples 1 to 7 in enlarged view.

### 5. DETAILED DESCRIPTION OF THE INVENTION

For the purposes of the present invention, the term “solid” shall mean that the fibre has a solid, not hollow or collapsed structure.

The term “standard” shall mean that the fibre is a regenerated cellulosic fibre obtained by the viscose process having a breaking force in the conditioned state Bc [cN] of less than 1.3·\sqrt{T}+2T and a force required to produce an elongation of 5% in the wet state Bm [cN] of less than 0.5·\sqrt{T} where T is the mean linear density in dtex.

The cross section of the fibre according to the invention roughly resembles a triangular shape. This triangular shape can be best defined by comparing the area of the fibre cross section with area of the largest equilateral triangle inscribed into said cross section.

The smaller the difference between the area of the cross section of the fibre and the area of this largest inscribed triangle, the more resembles the cross section of the fibre a triangular shape.

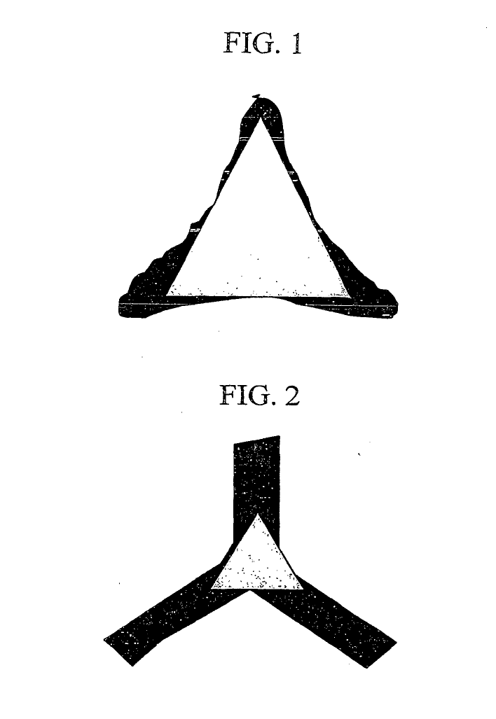

As can be seen from FIG. 1, in the case of a roughly triangular shape of the cross section, the area of the cross section of the fibre is not much larger than the area of the largest equilateral triangle inscribed into said cross section.

However, according to FIG. 2, if a triangle is inscribed into a fibre with a trilobal cross section having limbs with an aspect ratio of greater than 2:1, the area of the cross section of the fibre is much larger than the area of the inscribed triangle.

According to the present invention, the ratio between the area of the cross section of the fibre and the area of the largest inscribed equilateral triangle should be less than a factor of 2.50 times, preferably less than 2.40, especially preferred less than 2.25, said area of the cross section and said factor being determined according to the methods set out in detail below. For the purposes of the present invention this factor shall be called “Delta ratio”.

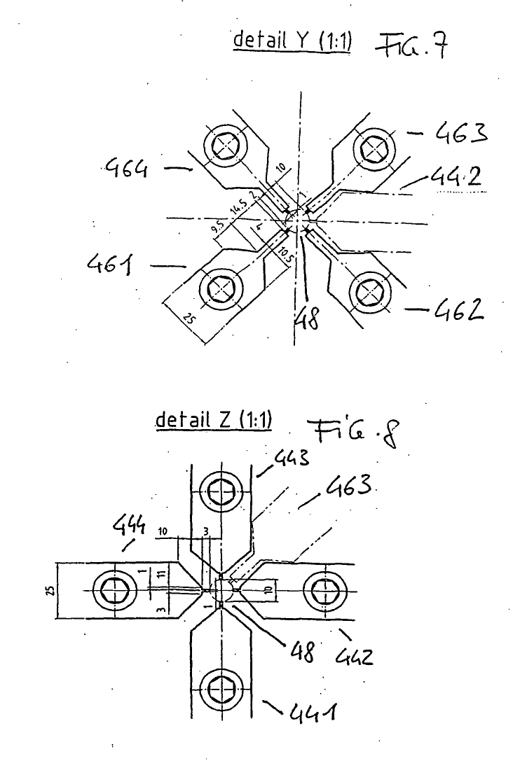

The article “Verzug, Verstreckung und Querschnittsmodifizierung” by Dr. Erich Treiber, Chemiefasem 5/1967, 344-348 discloses high wet modulus (HWM) filaments having been produced with a trilobal spinneret. The titre of the filament was 3.3 den, the tenacity 4 g/den in conditioned state and 2.4 g/den in wet state and the elongation was 10% resp. 14%. The cross section of this filament is depicted in FIG. 8a) of this publication and represents a Delta ratio of 1.67.

The Treiber publication is silent about the absorbent properties of such filaments. In general the skilled artisan would expect, that the water retention value of HWM fibres is significantly lower than that of viscose fibres.

The fibre according to the invention is preferably present in the form of staple fibre.

The titre of the fibre may be in the range of from 0.5 dtex to 6.0 dtex, preferably of from 2.5 dtex to 4 dtex.

Although the fibre according to the invention is a solid fibre and does not have limbs with an aspect ratio of more than 2:1 as disclosed in EP-A1 0 301 874, the fibre shows superior absorbency properties:
a specific Syngina absorbency of more than 6.0 g/g according to the test method disclosed below
a water retention value measured according to DIN 53814, using the Wt calculation scheme, of from 70 to 110%, preferably of from 80 to 90%.

The fibre according to the invention, furthermore, is less fragile than trilobal fibres due to its characteristic cross section and exhibits excellent processability during carding.

The fibre according to the invention is perfectly suitable for absorbent products, such as a tampon. Therefore, the present invention also provides an absorbent product, such as a tampon, including the fibre according to the invention in staple form.

It has been found that the fibre according to the invention can be produced with a process comprising the steps of
spinning a standard viscose spinning dope through a spinneret comprising spinning holes into a regenerating bath thereby forming filaments,
said spinning holes having a multi-limbed orifice, preferably a three-limbed orifice
   the limbs of said orifice having an aspect ratio of lower than 3:1
said viscose spinning dope having a ripening index of 10-20° Hottenroth, preferably 12-16° Hottenroth, and
said viscose spinning dope containing 0.1-7 wt. %, preferably 2-6 wt. % based on cellulose of a cellulose modifier
said regenerating bath containing
   from 70 to 100 g/l, preferably 75 to 85 g/l sulfuric acid,
   from 240 to 380 g/l, preferably 270 to 300 g/l sodium sulphate,
   from 5 to 30 g/l, preferably 7 to 12 g/l zinc sulphate and
said regenerating bath having a temperature of from 25 to 55° C., preferably 30 to 35° C.,
stretching and further treating said filaments according to known methods.

For the purposes of the present invention a standard viscose spinning dope is a state-of-the-art viscose solution, which is used to produce standard viscose fibres, typically characterised by a cellulose concentration of more than 7 wt. %, preferably more than 8%, an alkali ratio of less than 0.9, typically around 0.6, a gamma value below 50 and a ripening index of 20° Hottenroth and below.

Preferably the viscose modifier is a polyethylene glycol with a molecular weight of 600-3000, preferably 1200-1500.

All values given in wt. % in the present specification are calculated on the basis of the weight of cellulose.

In a preferred embodiment of the process, the filaments are treated with a fatty acid ester. Preferably the fatty acid ester used to treat the filaments is a polyoxyethylene sorbitan fatty acid ester such as TWEEN®20 (available from ICI Surfactants).

The filaments may be treated with the fatty acid ester in an amount of from 0.03 to 0.7% (w/w calculated on basis of cellulose), preferably of from 0.3 to 0.4%.

The stretching and further treatment of the filaments (such as cutting, finishing and drying) can be accomplished by methods known as such to the skilled artisan. Typically, the filaments are stretched after leaving said regenerating bath in a secondary bath and/or in air at a stretching ratio of from 40% to 90%, preferably 55% to 70%.

In the state-of-the-art process for the manufacturing of trilobal fibres, as disclosed e.g. in the experimental part of EP-AI 0301 874, regeneration conditions are applied which provide a rapid fixation of the fibre cross section in the same shape as extruded from the trilobal spinneret hole.

In the process according to the present invention however it is of critical importance, that the process parameters are balanced in that way, that the filaments extruded from a trilobal spinneret are regenerated slowly by allowing the filaments to contract the limbs towards the core, thus forming a substantially triangular cross section (referred to in the following as “Δ-shaped”).

For a certain viscose composition a slow regeneration process can e.g. be accomplished by dosage of a viscose modifier into the spinning dope in combination with a reduction of the sulfuric acid concentration and a low temperature of the spinbath.

It can be demonstrated, that in order to obtain Δ-shaped fibres instead of Y-shaped fibres when spinning through spinnerets with trilobal orifices, especially the combination of a modifier and a slow regenerating spinbath appears to be essential.

Without addition of a modifier, the cross section of the fibres obtained from a trilobal spinneret will be Y-shaped, even if the sulfuric acid concentration and temperature of the spinbath are reduced.

However, if a certain amount of modifier, e.g. PEG 1500, is added to the viscose spinning dope, the cross section of the fibres spun under the same conditions will be a Δ-shaped cross section.

On the other hand, if a conventional spinbath with higher sulfuric acid concentration is applied, the resulting fibres will have a Y-shaped cross section irrespective of whether a modifier is used or not used.

By using spinnerets the orifices of which have three limbs with a rather low aspect ratio, such as only slightly above 2:1 or below 2:1, it is possible to produce Δ-shaped fibres even if a spinbath with higher sulfuric acid concentration is applied.

Alternatively, the fibres according to the present invention can be produced by using spinnerets with triangular holes instead of trilobal spinnerets. In this case the regeneration conditions have to be adjusted in a way suitable to preserve the triangular cross section of the fibre. It has been found that spinning of a standard viscose spinning dope with an alkali ratio of 0.6 by means of a spinneret with equilateral triangular holes into a conventional spinbath or a slow regenerating spinbath with or without addition of polyethylene glycol as a modifier does not lead to fibres with a Delta ratio below 2.50 and a Syngina absorbency of more than 6 g/g fibre. However, if a viscose of high alkali ratio is spun by means of a triangular spinneret into a spinbath containing a high concentration of zinc sulphate, Δ-shaped fibres according to the present invention can be obtained.

Surprisingly it was found, that fibres with a triangular cross section as defined above show a significantly better Syngina absorbency than regular viscose fibres, up to the same level or even better than trilobal viscose fibres although their cross section does not comprise limbs with an aspect ratio of more than 2:1. The fibre according to the invention, furthermore, offers significant advantages in carding and tampon manufacturing due to its compact shape.

Test Methods

Taking of Photomicrograph of Fibre Cross Section

A bundle of parallelised staple fibres is threaded through a hole with a diameter of 1-2 mm in a stainless steel plate. The protruding fibres are cut off by means of a razor blade parallel to the surface of the steel plate. The plate is put under a microscope and a microphotograph of the fibre cross section is taken at a magnification of 1070:1.

Determination of Largest Inscribed Equilateral Triangle and Determination of Factor Between Area of Cross Section of the Fibre and Area of the Largest Inscribed Triangle

This determination can be performed on a personal computer via graphical software known as such and custom-made calculation software as described below.
1) The profile of a single fibre is transferred into a bitmap with color values identifying the fibre against the background. Only such fibre profiles are selected for graphical evaluation, which are completely visible and can be easily isolated from the neighbouring profiles, and which have a high contrast against the background. The fibre cross section is covered with a coarse grid. The grid size is approximately the twentieth part of the profile width and height respectively.
2) For a fixed angle between height and y-axis, the largest triangle with centroid C that can be inscribed in the profile is determined by continuously increasing the side length of the triangle until the boundary of the profile is reached.
3) The largest triangle is calculated for all centroids on the grid as in 2) and for all angles from 0 to 360° in steps of 0.5° and the centroid which corresponds to the maximum triangle is determined. Then for all pixels (x,y) in the surrounding area of this point, the largest triangle with centroid (x,y) is determined, yielding the maximum equilateral triangle that can be inscribed.
4) The area of the fibre cross section is determined by the colour pixels identifying the fibre. The area of the optimum equilateral triangle is determined according to 3). The Delta ratio is now obtained by dividing the area of the fibre cross section by the area of the optimum equilateral triangle.

The procedure is repeated for a total number of 12 individual fibres of the same Sample and the average Delta ratio is calculated.

Syngina Test:

The Syngina Test assesses the absorbency of fibres in a tampon. The test as described below is a simplified version of the EDANA Test method ERT 350.0-02.

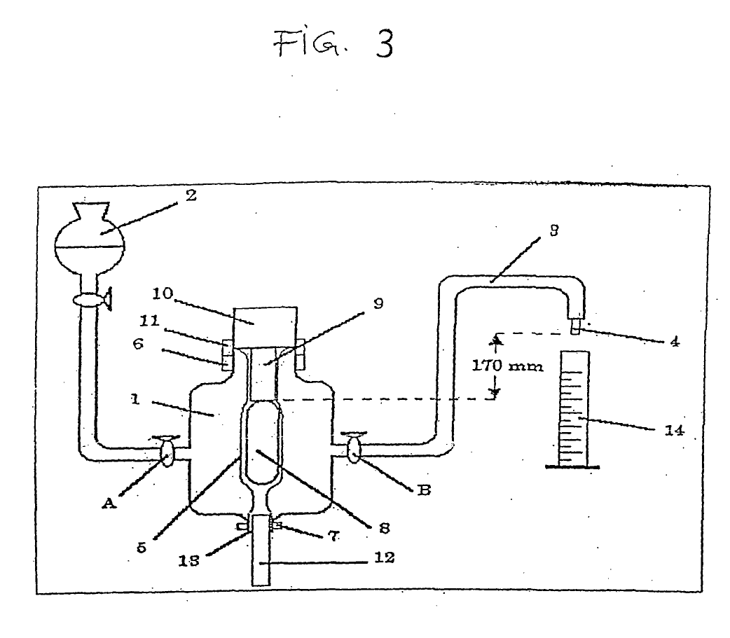

FIG. 3 shows the apparatus used to perform the test method, wherein

1 denotes the measuring cell

2 denotes a supplying vessel

3 denotes an overflow line

4 denotes a run out

5 denotes a condom

6,7,11 and 13 denote rubber rings, respectively

8 denotes a tampon or tampon-shaped plug

9 denotes a tube

10 denotes a filling tube

12 denotes a run out and

14 denotes a mensur.

A, B denote valves

The principle of the test method is to simulate the vaginal environment in the laboratory by applying standard pressure to a tampon inside a flexible membrane, being formed by a condom.

By introducing a certain amount of fluid until the tampon leaks, also the water retention and liquid absorptive capacity and water displacement can be measured. The tampon weight is taken before (dry) and after the test (wet) to calculate the weight of fluid absorbed.

Reagents

As a Syngina fluid, distilled or de-ionised water is used.

Preparation of Specimen

2.75 g of staple fibre with a humidity of 8-11% are weighed and fed into a carding machine type USTER MDTA 3, equipped with a rotoring 3. The speed of the combing roller is 1390 rpm. Each run takes 75 s. The resulting card sliver, which is about 90 cm long, is tripled to form a band with a length of 30 cm, which is pressed between 2 rollers or compacted on a calendar. Application of too high pressure during compacting of the card sliver may lead to the formation of a stiff, cardboard-like material, which has to be avoided.

The weight of the compacted sliver is adjusted to 2.70 g and put into a device to form a cylinder by winding. During this procedure the roll is weighed down by a 150 g counter cylinder.

The sample is then put into a mechanical press for plugs. This is a mechanical device, which is able to form tampon-shaped plugs. The plugs have the same volume, mass and fibre orientation as a commercial digital tampon, including 8 grooves along the side of the cylinder. The plug is pressed with 110 Nm for 10 minutes and is weighed again for redundance immediately before testing.

In FIGS. 4 to 8, the press by means of which the tampon-shaped pressed articles for carrying out the Syngina test are produced is illustrated.

The press 41 is arranged on a base plate 42 and consists of a rigidly installed lower bracing 43, in which a lower gripping device 44 is located, and an upper bracing 45 that is pivotable horizontally and vertically displaceable by means of a lifting device 47 and to which an upper gripping device 46 is connected.

FIG. 5 shows a section through the upper gripping device 46 according to lines A-A in FIG. 4. The upper gripping device comprises four upper gripping jaws 461-464.

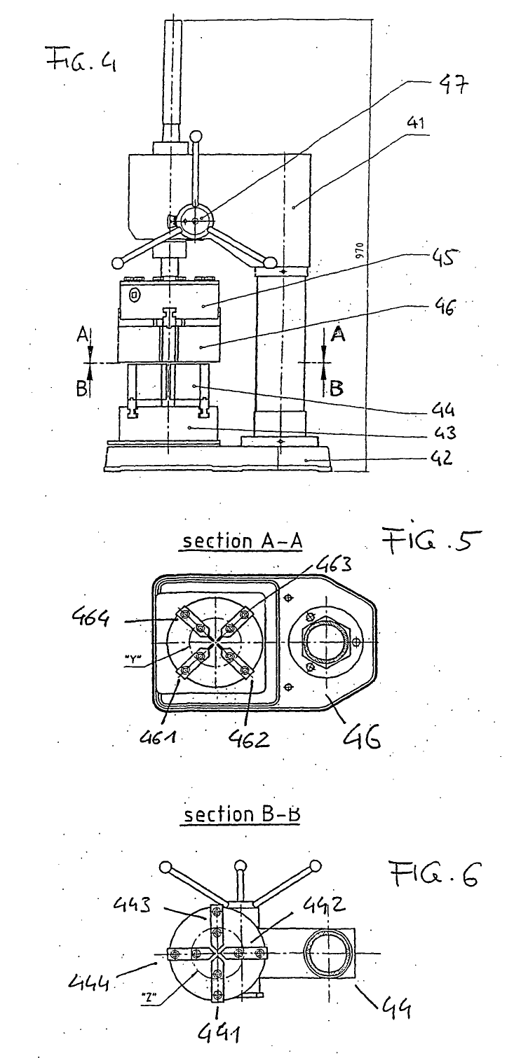

FIG. 6 shows a section through the lower gripping device 44 according to lines B-B in FIG. 4. The lower gripping device comprises four lower gripping jaws 441-444.

FIG. 7 shows an enlarged section of region Y in FIG. 5. The exact dimensions of the four upper gripping jaws 461-464 result from the dimensions in FIG. 7 (in mm). A lower gripping jaw 442 is illustrated by a segmented line.

FIG. 8 shows an enlarged section of region Z in FIG. 6. The exact dimensions of the four lower gripping jaws 441-444 result from the dimensions in FIG. 8 (in mm). An upper gripping jaw 463 is illustrated by a segmented line.

In order to prepare the sample, the previously prepared compressed and coiled card sliver (weight=2.7 g) is vertically introduced into the opening between the lower gripping jaws 441-444 and is fixed by the aid of a dynamometric key by applying a slight pressure on the lower gripping jaws. Subsequently, the upper gripping device 46 is swivelled in and brought down until the lower gripping jaws 441-444 and the upper gripping jaws 461-464 are flush with each other and—such as can be seen in FIGS. 7 and 8—end up lying alternately adjacent to each other. The coiled card sliver now is located in the space 48 (see FIGS. 7 and 8) that is predefined by the gripping jaws 441-444 and 461-464, respectively, and subsequently is pressed by the tightening of the gripping jaws. For that purpose, a dynamometric key is put into a square neck (not shown) provided in the lower bracing 43 and is tightened so strongly that a torque of 110 Nm is reached. The pressing operation lasts for 10 minutes. In that way, the pressed article receives its characteristic shape with 8 grooves.

This plug can be used for the Syngina test without further modification. The length of the plug is about 53 mm, its diameter is 14-15 mm; it does not change its longitudinal or radial dimension for at least 7 days.

If a tampon is used as a specimen, the wrapping or the applicator have to be removed. The test specimen should be unwrapped immediately before testing, and the withdrawal cord should be cut away.

The number of specimens per test should be three.

Condom Installation and Replacement

A straight unlubricated condom having a tensile strength between 17 MPa and 30 MPa is used as a test membrane. The condom is opened and unraveled. The condom is marked at 20 mm and 160 mm length from the open end.

The condom is inserted through the chamber 1 of the test apparatus (FIG. 3) with the aid of a rod, so that the 160 mm mark rests on the edge of the smaller opening of the chamber 1 (bottom of chamber 1).

The tip of the condom is cut and secured with a rubber band such that the 160 mm mark remains on the edge of the smaller opening of chamber 1.

The condom is drawn through the large opening of chamber 1 so that the 20 mm mark rests on the opening's edge and is secured there with a rubber band.

Test condoms are replaced (a) if they leak, (b)—monthly—whichever applies first.

Procedure

The tampon or the pressed plug prepared according to section “Preparation of Specimen” above is weighed to the nearest 0.01 gram. The weight is recorded.

While chamber 1 of the test apparatus is empty, tampon 8 is placed within the condom 5 as shown in FIG. 3 so that the centre of the tampon is at the centre of chamber 1 and the bottom end (end where withdrawal cord is located) is positioned toward the bottom of chamber 1. It is helpful to use tweezers to place the plug in the center of this cell.

After this, valve A is opened so that chamber 1 is filled with water. A small tube 9 is inserted into the chamber 1, so that it contacts the top end of tampon/plug 8. Valve A is closed again.

Then, valve B is opened for pressure equalisation (a pressure equivalent to 170 mm water column is established as can be seen from FIG. 3). Filling tube 10 is inserted with a rubber ring 11. 25 ml test liquid is filled into tube 10. A stop watch is started.

After 3 minutes valve B is closed (except there is still some water replaced via run out 4). If any liquid is standing over the filling tube 10 and small tube 9, it is sucked off with a Socorex pipette. Filling tube 10 is removed, and the measuring cell is raised.

Tube 9 is removed, valve A is opened and the condom is relieved, which makes it easy to remove tampon/plug 8 with tweezers. Afterwards valve A is closed, and chamber 1 is fixed.

The removed tampon/plug is weighed immediately to the nearest 0.01 gram. The wet weight is recorded. The remaining water is drained from chamber 1.

The test should be repeated three times with a new plug from the same fibre sample.

For the test, chamber 1 should be filled without any bubbles.

Calculation and Expression of Results:

The absorbency of each specimen tampon/plug is calculated as follows:
A=B−C, wherein

A=Absorbency of the tampon/plug in grams

B=Weight in grams of the saturated (wet) tampon/plug

C=Weight in grams of dry tampon/plug

The results are expressed to the first decimal. The average absorbency of the total number of test specimens is calculated.

The specific Syngina absorbency in g test liquid/g fibre is calculated by dividing the average absorbency (A) by the average weight of the dry tampons/plugs (C) in grams.

Water Retention

Water retention of the fibres is measured according to the test method described in DIN 53814, using the Wt calculation scheme.

Water Holding Capacity

Water holding capacity of the fibres is measured according to the test method for absorbency of Viscose waddings, Absorbent described in European Pharmacopoeia 4 01/2002:0034.

### 6. WORKING EXAMPLES

Preferred embodiments of the invention are explained in more detail by way of the following examples.

### Example 1

### Δ-Shaped Fibre

A viscose containing 8.70% Cellulose, 5.20% alkali and 2.3% sulphur having a ripening index of 14.2° Hottenroth and a viscosity of 58 ball fall seconds (bfs, determined according to Zellcheming-Merkblatt III/5/E) was spun into a regeneration bath containing 76.5 g/l sulphuric acid, 272 g/l sodium sulphate and 10.4 g/l zinc sulphate at a temperature of 32° C. by means of a trilobal spinneret. The spinneret had 625 trilobal holes with 3 limbs of 72×33 μm (aspect ratio: 2.18). Before spinning, 5 wt. % of an aqueous solution of polyethylene glycol 1500 were added to the viscose.

The spinning speed was 50 m/min. The filaments were stretched by 55% in a hot secondary bath containing 17 g/l sulphuric acid, cut into staples of 40 mm length, washed, desulphurized, bleached, finished at 70° C. and a pH of 5 with 10 g/l of a polyoxyethylene sorbitan fatty acid ester (Tween 20, available from ICI surfactants) and dried.

The fibres had a titre of 3.0 dtex, a water retention value of 103% and a water imbibition of 23.8 g/g. The Syngina absorbency according to the Syngina Test as described above was 6.7 g/g. The Delta ratio of the fibre was 2.26. Its typical shape is depicted in FIG. 9.

### Comparison Example 2

### Spinning without Use of Modifier

A viscose fibre was spun under the same conditions as described in example 1 with the exception that no polyethylene glycol was added to the viscose.

The fibres had a titre of 2.9 dtex, the water retention value was 120% and the water imbibition was 24.8 g/g. The Syngina absorbency according to the Syngina Test as described above was 5.9 g/g.

The Delta ratio of the fibre was 4.85. Its trilobal shape is depicted in FIG. 10.

### Comparison Example 3

### High Sulfuric Acid Content in the Regeneration Bath

A viscose containing 8.65% Cellulose, 5.16% alkali and 2.3% sulphur having a ripening index of 14° Hottemoth and a viscosity of 63 bfs was spun into a regeneration bath containing 131 g/l sulphuric acid, 367 g/l sodium sulphate and 11 g/l zinc sulphate at a temperature of 49° C. by means of a trilobal spinneret as described in example 1. Before spinning, 2.5 wt. % of an aqueous solution of polyethylene glycol 1500 were added to the viscose.

The spinning speed was 50 m/min. The filaments were stretched by 76% in a hot secondary bath containing 19 g/l sulphuric acid, cut into staples of 40 mm length, washed, desulphurized, bleached, finished at 70° C. and a pH of 5 with 5 g/l of a polyoxyethylene sorbitan fatty acid ester (Tween 20, available from ICI surfactants) and dried.

The fibres had a titre of 3.2 dtex, a water retention value of 87% and a water imbibition of 23.7 g/g. The Syngina absorbency according to the Syngina Test as described above was 6.6 g/g.

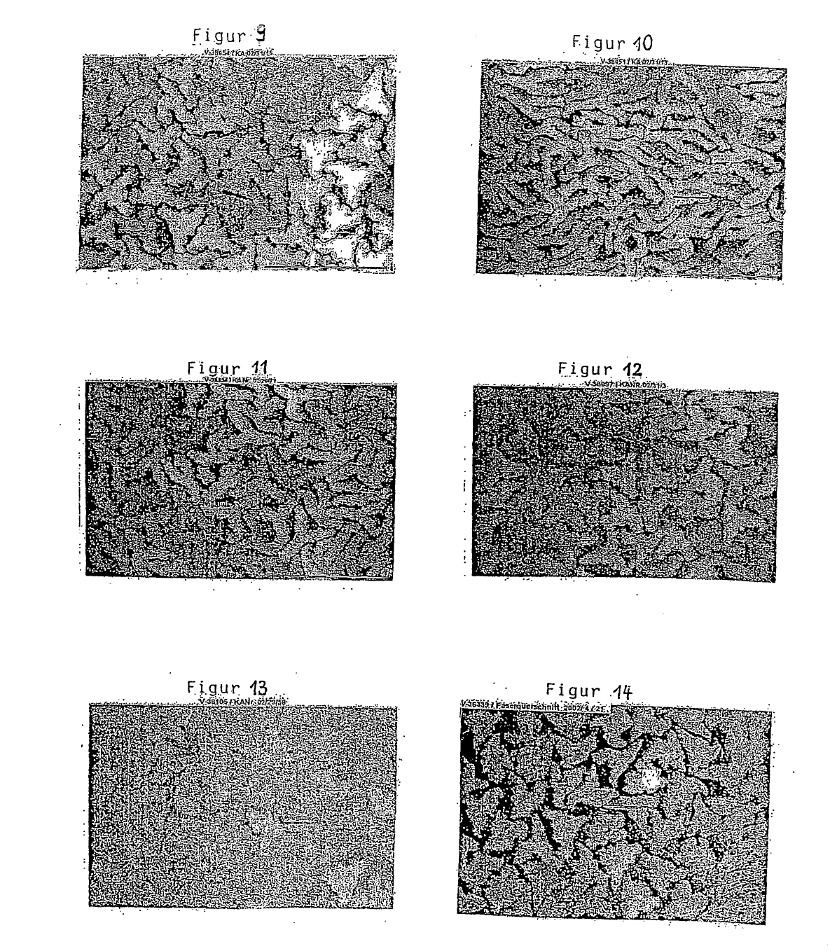

The Delta ratio of the fibre was 4.07. Its typical shape is depicted in FIG. 11.

### Example 4

### Δ-Shaped Fibre

A viscose containing 8.65% Cellulose, 5.14% alkali and 2.3% sulphur having a ripening index of 13.60 Hottemoth and a viscosity of 65 bfs was spun into a regeneration bath containing 85 g/l sulphuric acid, 276 g/l sodium sulphate and 11 g/l zinc sulphate at a temperature of 31° C. by means of a trilobal spinneret as described in example 1. Before spinning, 5 wt. % of an aqueous solution of polyethylene glycol 1500 were added to the viscose.

The spinning speed was 50 m/min. The filaments were stretched by 55% in a hot secondary bath containing 19 g/l sulphuric acid, cut into staples of 40 mm length, washed, desulphurized, bleached, finished at 70° C. and a pH of 5 with 10 g/l of a polyoxyethylene sorbitan fatty acid ester (Tween 20, available from ICI surfactants) and dried.

The fibres had a titre of 2.9 dtex. The Syngina absorbency according to the Syngina Test as described above was 6.9 g/g.

The Delta ratio of the fibre was 1.91. Its typical shape is depicted in FIG. 12.

### Example 5

### Δ-Fibre, Short-Limbed Trilobal Spinneret

A viscose containing 8.67% Cellulose, 5.15% alkali and 2.3% sulphur having a ripening index of 15° Hottenroth and a viscosity of 62 bfs was spun into a regeneration bath containing 85 g/l sulphuric acid, 277 g/l sodium sulphate and 11 g/l zinc sulphate at a temperature of 53° C. by means of a trilobal spinneret. The spinneret had 625 holes, each hole having 3 limbs with 45×33 μm (aspect ratio: 1.36) on an equilateral triangular core. The radius of the circumscribed circle is 80 μm. Before spinning, 5 wt. % of an aqueous solution of polyethylene glycol 1500 were added to the viscose.

The spinning speed was 50 m/min. The filaments were stretched by 55% in a hot secondary bath containing 17.6 g/l sulphuric acid, cut into staples of 40 mm length, washed, desulphurized, bleached, finished at 70° C. and a pH of 5 with 5 g/l of a polyoxyethylene sorbitan fatty acid ester (Tween 20, available from ICI surfactants) and dried.

The fibres had a titre of 3.2 dtex, a water retention value of 78.5% and a water imbibition of 18.6 g/g. The Syngina absorbency according to the Syngina Test as described above was 6.1 g/g.

The Delta ratio of the fibre was 1.63. Its typical shape is depicted in FIG. 13.

### Example 6

### Δ-Fibre Triangular Spinneret

A viscose containing 8.23% Cellulose, 7.15% alkali and 2.20% sulphur having a ripening index of 14.5° Hottenroth and a viscosity of 52 bfs was spun into a regeneration bath containing 98 g/l sulphuric acid, 351 g/l sodium sulphate and 28.2 g/l zinc sulphate at a temperature of 49° C. by means of a triangular spinneret. The spinneret had 625 holes, each hole having the shape of an equilateral triangle with s=129 μm.

The spinning speed was 55 m/min. The filaments were stretched by 82% in a hot secondary bath containing 19.6 g/l sulphuric acid, cut into staples of 40 mm length, washed, desulphurized, bleached, finished at 70° C. and a pH of 5 with 10 g/l of a polyoxyethylene sorbitan fatty acid ester (Tween 20, available from ICI surfactants) and dried.

The fibres had a titre of 2.96 dtex. The Syngina absorbency according to the Syngina Test as described above was 6.4 g/g.

The Delta ratio of the fibre was 2.03. Its typical shape is depicted in FIG. 14.

### Comparison Example 7

### Trilobal Fibre with 89×25 μm-Spinneret (Aspect Ratio 3.56)

A viscose containing 8.80% Cellulose, 5.20% alkali having a ripening index of 13.5° Hottenroth and a viscosity of 70 bfs was spun into a regeneration bath containing 76 g/l sulphuric acid, 266 g/l sodium sulphate and 10.4 g/l zinc sulphate at a temperature of 30° C. by means of a trilobal spinneret. The spinneret had 625 trilobal holes with 3 limbs of 89×25 μm (aspect ratio: 3.56). Before spinning, 5 wt. % of an aqueous solution of polyethylene glycol 1500 were added to the viscose.

The spinning speed was 50 m/min. The filaments were stretched by 55% in a hot secondary bath containing 17 g/l sulphuric acid, cut into staples of 40 mm length, washed, desulphurized, bleached, finished at 70° C. and a pH of 5 with 10 g/l of a polyoxyethylene sorbitan fatty acid ester (Tween 20, available from ICI surfactants) and dried.

The fibres had a titre of 2.97 dtex, and a water imbibition value of 25.0 g/g. The Syngina absorbency according to the Syngina Test as described above was 6.8 g/g.

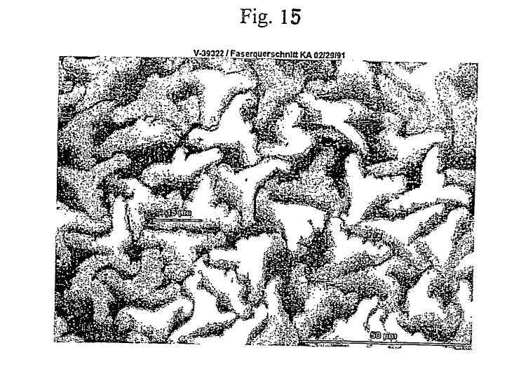

The Delta ratio of the fibre was 2.64. Its typical shape is depicted in FIG. 15.

1. A solid regenerated standard viscose fibre having a cross section the area of which is larger than the area of the largest equilateral triangle inscribed into said cross section by a factor of less than 2.50 times, and having a Syngina absorbency as defined hereinbefore of more than 6.0 g/g fibre.

2. A fibre according to claim 1, in the form of staple fibre.

3. A fibre according to claim 1 or 2, having a titre of from 0.5 dtex to 6.0 dtex.

4. A fibre according to claim 1 or 2, having a water retention value measured according to DIN 53814 of from 70 to 110%.

5. A process for the manufacture of a solid regenerated standard viscose fibre, comprising:
spinning a standard viscose spinning dope through a spinneret comprising spinning holes into a regenerating bath thereby forming filaments, wherein
(i) said spinning holes comprise a multi-limbed orifice the limbs of said orifice having an aspect ratio of lower than 3:1;
(ii) said viscose spinning dope has a ripening index of 10-20° Hottenroth, and
(iii) said viscose spinning dope contains 0.1-7 wt. %, based on cellulose of a viscose modifier;
(iv) said regenerating bath contains
from 70 to 100 g/l sulfuric acid,
from 240 to 380 g/l sodium sulphate, and
from 5 to 30 g/l zinc sulphate; and
(v) said regenerating bath has a temperature of from 25 to 55° C., and
(b) stretching and further treating said filaments.

6. A process according to claim 5, wherein the viscose modifier is a polyethylene glycol with a molecular weight of 600-3000.

7. A process according to claim 5 or 6, wherein said filaments are treated with a fatty acid ester.

8. A process according to claim 5 or 6, wherein said filaments are treated with a polyoxyethylene sorbitan fatty acid ester.

9. An absorbent product, comprising a fibre according to claim 2.

10. A solid regenerated standard viscose fibre according to claim 1 or 2 wherein said factor by which the area of the cross section is larger than the area of the largest equilateral triangle inscribed into said cross section is less than 2.40 times.

11. A solid regenerated standard viscose fibre according to claim 1 or 2 wherein said factor by which the area of the cross section is larger than the area of the largest equilateral triangle inscribed into said cross section is less than 2.25 times.

12. An absorbent product, comprising a fibre according to claim 2 having a titre of from 0.5 dtex to 6.0 dtex.

13. A tampon, comprising a fibre according to claim 2 having a titre of from 0.5 dtex to 6.0 dtex.

14. An absorbent product, comprising a fibre according to claim 2 having a water retention value measured according to DIN 53814 of from 70 to 110%.

15. A tampon, comprising a fibre according to claim 2 having a water retention value measured according to DIN 53814 of from 70 to 110%.

16. A process for the manufacture of a solid regenerated standard viscose fibre, comprising:
(a) spinning a standard viscose spinning dope through a spinneret comprising spinning holes into a regenerating bath thereby forming filaments, wherein
(i) said spinning holes comprise a multi-limbed orifice, the limbs of said orifice having an aspect ratio of about 3:1 or less;
(ii) the viscose spinning dope has a ripening index of 10-20° Hottenroth;
(iii) the viscose spinning dope contains 0.1-7 wt. % based on cellulose of a viscose modifier; and
(iv) the regenerating bath contains
from 75 to 85 g/l sulfuric acid,
from 270 to 300 g/l sodium sulphate, and
from 7 to 12 g/l zinc sulphate; and
(v) the regenerating bath has a temperature of from 25 to 55° C.; and
(b) stretching and further treating said filaments.

17. A process for the manufacture of a solid regenerated standard viscose fibre, comprising:
(a) spinning a standard viscose spinning dope through a spinneret comprising spinning holes into a regenerating bath thereby forming filaments, wherein
(i) said spinning holes having a three-limbed orifice, the limbs of said orifice having an aspect ratio of about 3:1 or less,
(ii) the viscose spinning dope has a ripening index of 12-16° Hottenroth, and
(iii) contains 2-6 wt. % based on cellulose, of a viscose modifier
(iv) the regenerating bath contains
from 75 to 85 g/l sulfuric acid,
from 270 to 300 g/l sodium sulphate, and
from 7 to 12 g/l zinc sulphate; and
(iv) the regenerating bath having a temperature of from 30 to 35° C.,
(b) stretching and further treating said filaments.

18. A process according to claim 16 or 17, wherein the viscose modifier is a polyethylene glycol with a molecular weight of 600-3000.

19. A process according to claim 16 or 17, wherein said filaments are treated with a fatty acid ester.

20. A process according to claim 16 or 17 wherein said filaments are treated with a polyoxyethylene sorbitan fatty acid ester.
